(19) United States
(12) Patent Application Publication
(10) Pub. No.: US 2008/0039837 A1
(43) Pub. Date: Feb. 14, 2008

(54) METHOD AND APPARATUS FOR TREATMENT OF BONES

(76) Inventor: Michael A. Gambale, Nowell, MA (US)

Correspondence Address: TAROLLI, SUNDHEIM, COVELL & TUMMINO L.L.P., SUITE 1700, 1300 EAST NINTH STREET, CLEVELAND, OH, 44114, US

(21) Appl. No.: 11/835,836

(22) Filed: Aug. 8, 2007

Related U.S. Application Data
(60) Provisional application No. 60/836,802, filed on Aug. 10, 2006.

(51) Int. Cl. A61B 17/58; A61B 17/56

(52) U.S. Cl. 606/60; 606/73

(57) ABSTRACT

An apparatus for use in attaching bone in a patient's body is provided having first and second plates, each of the plates at least partially defining a recess. A nut is also provided, having an internal thread convolution and a plurality of bendable sections. A screw is also provided, having an external thread convolution which, upon rotation of the screw, engages with the internal thread convolution of the nut; and a plurality of resiliently bendable sections. Further rotation of the screw retains both the bendable sections of the screw in the recess of the first plate and the bendable sections of the nut in the recess of the second plate, prohibiting relative movement between the screw and the first plate and the nut and the second plate and providing radial compression to the first and second plates to secure bones together.

### RELATED APPLICATION

This application claims priority from U.S. Provisional Patent Application Ser. No. 60/836,802, filed on Aug. 10, 2006, the subject matter of which is incorporated herein by reference.

### TECHNICAL FIELD

The present invention relates to a method and apparatus for treating bone and more specifically, to a method and apparatus for adjacent sections of bones.

### BACKGROUND OF THE INVENTION

During the treatment of bones in a patient's body, it is often necessary to remove a section of the bone to access the tissue or organs underneath them. This is especially true in a craniotomy, in which a portion of the cranial vault is removed or folded back in a flap to permit surgical access to the cranial contents (such as the brain). To perform the craniotomy, several burr holes are drilled through the skull. The number and position of these holes varies depending on the shape of bone to be removed. For example, three burr holes are drilled at corner points if a triangular bone flap is desired. The burr holes are then connected by osteotomy cuts, for example, by using a flexible saw which is passed internally between the burr holes. The saw is then oscillated back and forth to cut the skull between the burr holes and allow the flap to be removed from the skull.

Once the appropriate procedures have been performed on the brain, it is necessary to return the cranial flap back to the skull. It is imperative that the flap be secured in the appropriate spot and affixed rigidly to the skull to ensure there is no relative movement between the two pieces for sufficiently long enough that adequate bone bridging and regeneration can occur. To accomplish this, varying devices employing several types of plates and screws have been devised to secure the flap to the skull. However, once implanted into the patient, the devices are susceptible to vibration as the patient goes through day-to-day life. After extended periods of time undergoing such vibrations, the screws holding the plates down between the cranial flap and the skull can begin to unscrew, loosening the plate and allowing the cranial flap to move relative to the skull. This movement and weakened fixture impedes the ability of the body's osteoclasts to deposit new bone between the cranial flap and the skull, and thus retards healing and may ultimately render the new bone weaker and more susceptible to continued or future fracture.

The present invention alleviates this problem by utilizing screw heads that are ultimately locked into position relative to the fixation plates that hold the cranial flap to the skull. The nature of the heads creates an outward axial force against the plates they are employed in, which prohibits unscrewing of the screw during extended vibration of the patients skull due to the patient's day-to-day activity. The inability of the screws to loosen from the fixation plates allows the device to maintain a rigid connection between the cranial flap and the skull, facilitating bone regeneration along the cut lines of the saw and allowing the cranial flap to adequately heal to the skull.

### SUMMARY OF THE INVENTION

In accordance with the present invention, an apparatus for use in attaching bone in a patient's body is provided having first and second plates, each of the plates has a flange portion and a trunk portion. The flange portion has an outer face and an inner face, and the trunk portion has an outer wall and an end. The flange portion at least partially defines a recess. The first and second plates have an opening which extends from the recess to the end wall of the trunk portion. A nut is also provided, having a head portion and a body portion. The body portion has a opening having an internal thread convolution, and the head portion has a plurality of resiliently bendable sections which extend outward from the head portion. A screw is also provided, having a head portion and a shank portion. The shank portion has an external thread convolution which is engageable with the internal thread convolution of the nut, and the head portion has a plurality of resiliently bendable sections which extend outward from the head portion. The screw is rotatable to turn the external thread convolution on the shank portion into engagement with the internal thread convolution of the nut. Furthermore, the bendable sections of the screw are movable into engagement with the recess of the first plate upon rotation of the screw, wherein further rotation of the screw retains the bendable sections in the recess of the first plate against rotation relative to the first plate, and the bendable sections of the nut move into engagement with the recess of the second plate as the nut becomes engaged with the screw. The bendable sections of the nut become retained in the recess of the second plate against rotation relative to the second plate, and the bendable sections of the screw and the bendable sections of the nut provide radial compression to the first and second plate upon engagement to secure bones together.

In accordance with another embodiment of the present invention, an apparatus for use in attaching bone in a patient's body comprises first and second plates. Each of the plates has a flange portion and a trunk portion, the flange portion has an outer face and an inner face, and the trunk portion has an outer wall and an end. The flange portion at least partially defines a recess extending from the outer face toward the inner face, and having a bottom surface which is spaced from the inner face. The recess further has a side surface which extends transverse to the outer face and to the bottom surface of the recess. The first and second plates have an opening which extends from the bottom surface of the recess to the end wall of the trunk portion. A nut is also provided, having a head portion and a body portion. The body portion has a opening at a leading end, the opening having an internal thread convolution extending from the leading end of the body portion towards the head portion. The head portion has a plurality of resiliently bendable sections which extend outward from a central portion of the head portion in a direction toward the leading end of the body portion when the nut is in an initial condition, wherein each of the resiliently bendable sections has an end portion disposed at a distal end. A screw is also provided, having a head portion and a shank portion. The shank portion has an external thread convolution extending from a leading end of the shank portion towards the head portion which is engageable with the internal thread convolution of the nut. The head portion has a plurality of resiliently bendable sections which extend outward from a central portion of the head portion in a direction toward the leading end of the shank portion when the screw is in an initial condition, wherein each of the resiliently bendable sections has an end portion disposed at a distal end. The screw is rotatable about a longitudinal central axis of the shank portion to turn the external thread convolution on the shank portion into engagement with the internal thread convolution of the nut. The resiliently bendable sections of the screw move into engagement with the bottom surface of the recess of the first plate and are deflected in a direction away from the leading end of the shank portion of the screw as the screw is rotated about the longitudinal central axis. The end portions of the resiliently bendable sections of the screw move into engagement with the side surface of the recess of the first plate as the sections are deflected to retain the screw against rotation relative to the first plate. The resiliently bendable sections on the nut move into sliding engagement with the bottom surface of the recess of the second plate and are deflected in a direction away from the leading end of the body potion of the nut as the nut becomes engaged with the screw. The end portions of the resiliently bendable sections of the nut move into engagement with the side surface of the recess of the second plate as the sections are deflected to retain the nut against rotation relative to the second plate. The end portions of the resiliently bendable sections of the screw and the end portions of the resiliently bendable sections of the nut provide radial compression to the side surface of the first and second plate upon engagement of the screw and the nut to secure bones together.

A method of attaching bone in patient's body is further provided. A first plate is placed near a top surface of a bone. The first plate at least partially defines a recess, and has an opening which extends from the recess to an end wall of the first plate. A second plate is placed near a bottom surface of the bone and the bone fragment. The second plate at least partially defines a recess and has an opening which extends from the recess to an end wall of the second plate. The distal end of a screw is inserted through the opening of the first plate. The screw has an external thread convolution at the distal end and bendable sections at the proximal end. The distal end of a nut is inserted through the opening of the second plate. The nut has an internal thread convolution at the distal end and bendable sections at the proximal end. The screw is rotated to engage the external thread convolution of the screw with the internal thread convolution of the nut, wherein further rotation of the screw clamps the bone and the bone fragment between the first plate and the second plate. The bendable sections of the screw are retained against the recess of the first plate, which prevents further rotation of the screw relative to the first plate; and the bendable sections of the nut are retained against the recess of the second plate, which prevents further rotation of the nut relative to the second plate.

In accordance with another embodiment of the present invention, an apparatus for use in attaching bone in a patient's body comprises a base. The base has a top surface, an end wall, and a side wall. The top surface at least partially defines a recess. The base further comprises an opening which extends from the recess toward the end wall. The opening defines an inner wall which has an internal thread convolution. A fastener which has a post and a trunk is also provided. The trunk has an external thread convolution which is engageable with the internal thread convolution of the base. The post has a plurality of resiliently bendable sections which extend outward from the post. The fastener is rotatable to turn the external thread convolution on the trunk into engagement with the internal thread convolution of the base. The bendable sections on the post are movable into engagement with the recess of the base upon rotation of the fastener. Further rotation of the fastener retains the bendable sections in the recess of the base against rotation relative to the base. The bendable sections of the fastener provide radial compression to the base upon engagement to secure bones together.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1A is a schematic illustration of a generic bone on which the present invention will be utilized;

FIG. 1B is an exploded side view showing an apparatus for use in attaching bone in a patient's body in accordance with the present invention;

FIG. 2A is a section view of the first plate of FIG. 1B;

FIG. 2B is a top view of the first plate of FIG. 2A;

FIG. 3A is a section view of the second plate of FIG. 1B;

FIG. 3B is a top view of the second plate of FIG. 3B;

FIG. 4A is a side view of the screw of FIG. 1B;

FIG. 4B is a top view of the screw of FIG. 4A;

FIG. 5A is a side view of the nut of FIG. 1B;

FIG. 5B is a sectional view of the nut of FIG. 5A;

FIG. 5C is a top view of the nut of FIG. 5A;

FIG. 6 is a section view of the screw inserted into the first plate, and the nut inserted into the second plate;

FIG. 7 is a section view of the screw inserted into the first plate and the nut inserted into the second plate, where the screw is engaged with the nut and the skull and skull fragment are positioned between the first and second plates;

FIG. 8 is an enlarged view of the screw and the first plate of FIG. 7;

FIG. 9A is section view of the present invention in its installed condition;

FIG. 9B is an enlarged view of the screw and the first plate of FIG. 9A;

FIG. 9C is a top plan view of the present invention of FIG. 9A;

FIG. 10 is a schematic illustration of the present invention in an installed condition.

FIG. 11 is a schematic illustration of a jaw bone on which a further embodiment of the present invention will be utilized;

FIG. 12 is an exploded side view showing an apparatus for use in providing a dental implantation site;

FIG. 13A is a side view of the base portion of FIG. 12;

FIG. 13B is a top view of the base portion of FIG. 13A;

FIG. 14A is a section view of the fastener portion of FIG. 12;

FIG. 14B is a top view of the fastener portion of FIG. 14A;

FIG. 15 is a section view of the fastener portion engaged with the base portion;

FIG. 16A is a section view of the fastener portion partially engaged with the base portion;

FIG. 16B is a section view of the fastener portion fully engaged with the base portion;

FIG. 16C is a top view of the present invention of FIGS. 16B; and

FIG. 17 is a schematic illustration of the present invention of FIG. 12 installed within the jaw.

### DETAILED DESCRIPTION

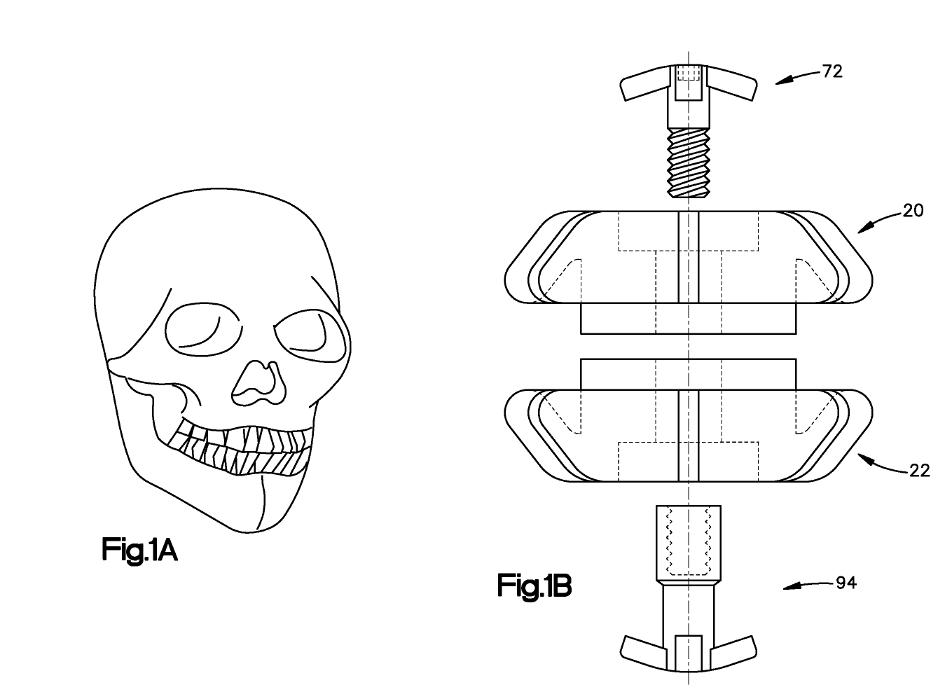

A bone disposed in a patient's body has been illustrated schematically in FIG. 1A. Although the bone illustrated in FIG. 1A is a skull 10, it should be understood that the skull is representative of many bones in a patient's body and that the present invention may be utilized in association with bones other than skull bones. As shown in the exploded view of FIG. 1B, the present invention utilizes a first plate 20, a second plate 22, a screw 72, and a nut 94 to secure the piece of bone 10 relative to another piece of bone, a skull fragment 14, to effectuate healing and bone regeneration.

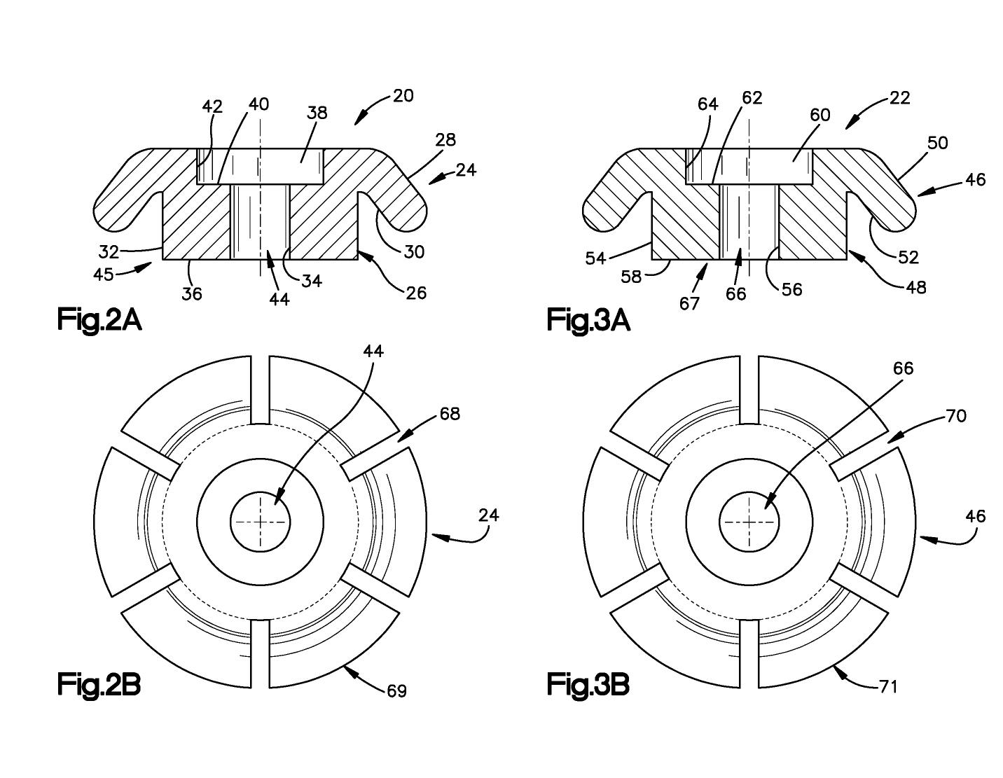

As shown in FIG. 2A, first plate 20 comprises a flange portion 24 and a trunk portion 26. The flange portion 24 and trunk portion 26 are substantially circular in nature. The flange portion 24 has a larger cross-sectional area than the trunk portion 26. In an initial condition, the flange portion 24 extends radially outward from the trunk portion 26 in a direction toward the distal end 45 of the plate. The plate 22 is made of any biocompatible material that is capable of allowing the flange portion 24 to move in a direction away from the trunk portion 26 when a force is applied in that direction.

The trunk portion 26 is sized to fit within a pertuator hole left within the skull 10 following surgery. The flange portion 24 includes an outer face 28 and an inner face 30. The outer face 28 is spaced approximately 0.01″ to 0.05″ apart from the inner face 30, but the distance is generally minimized so that the skin that is to be pulled back over the device once the bone is fixated will not be displaced too far away from the fixation site. This minimizes both discomfort to the patient and unsightly bumps and protrusions on the patient's scalp. The inner face 28 is oriented such that it will reside in substantial contact with the outer surface of the bone at the fixation site.

The flange portion 24 further includes a recess 38 defined from the outer face 28 towards the inner face 30. The recess 38 has a bottom surface 40 and a side surface 42. The bottom surface 40 is parallel to the outer face 28 of the flange portion 24, and the side surface 42 extends transverse to the outer face 28 and extends from the outer face 28 to the bottom surface 40. Although it is shown that the recess 38 has a circular profile, it is contemplated that the recess 38 could have any shaped profile, including triangular, rectangular, or any other multiple-sided configuration.

The trunk portion 26 includes an inner wall 34, an outer wall 32, an end wall 36, and an opening 44. The outer wall 32 is sized to fit within a pertuator hole left within the skull 10 following surgery. The inner wall 34 is co-axial with the outer wall 32 and extends transverse to the bottom surface 40 of the recess 38 from the bottom surface 40 to the end wall 36 of the trunk portion 26. The end wall 36 runs substantially parallel to the outer face 28. An opening 44 extends co-axially with the recess 38 and is defined between the bottom surface 40 of the recess 38 and the end wall 36 of the trunk portion 26. The opening 44 is circular in nature and generally of a smaller diameter than the recess 38.

As shown in FIG. 2B, the flange portion 24 further includes a plurality of expansion slots 68 extending from the periphery of the recess 38 to the periphery of the flange portion 24. The expansion slots 68 are about 0.01″ to 0.05″ wide, extend transverse to the outer face 28 of the flange portion 24, and are defined from the outer face 28 to the inner face 30. Although six expansion slots 68 are depicted in the drawing, it is contemplated that any number of slots 68 could be utilized. As will be further explained later, the expansion slots 68 allow the flange portion 24 to expand radially outward from the distal end 45 of the plate 20 to provide axial compression at the bone fixation site to aid in securing the bones together. The expansion slots 68 accomplish this by allowing the flange portion 24 to expand as a series of flange sections 69 instead of as a single flange. This configuration allows the flange portion 24 to provide a larger, and more stable, surface area of axial compression to the skull 10 and the skull fragment 14, as well as allowing the flange portion 24 to more precisely follow the contour of the skull 10 and the skull fragment 14.

As shown in FIG. 3A, the second plate 22 comprises a flange portion 46 and a trunk portion 48. The flange portion 46 and trunk portion 48 are substantially circular in nature. The flange portion 46 has a larger cross-sectional area than the trunk portion 48. In an initial condition, the flange portion 46 extends radially outward from the trunk portion 48 in a direction toward the distal end 67 of the plate 22. The plate 22 is made of any biocompatible material that is capable of allowing the flange portion 46 to move in a direction away from the trunk portion 48 when a force is applied in that direction.

The trunk portion 48 is sized to fit within the pertuator hole left within the skull 10 following surgery. The flange portion 46 includes an outer face 50 and an inner face 52. The outer face 50 is spaced approximately 0.01″ to 0.05″ apart from the inner face 52, but the distance is generally minimized so that the skin that is to be pulled back over the device once the bone is fixated will not be displaced too far away from fixation site. This minimizes both discomfort to the patient and unsightly bumps and protrusions on the patient's scalp. The inner face 52 is oriented such that it will reside in substantial contact with the outer surface of the bone at the fixation site.

The flange portion 46 further includes a recess 60 defined from the outer face 50 towards the inner face 52. The recess 60 has a bottom surface 62 and a side surface 64. The bottom surface 62 is parallel to the outer face 50 of the flange portion 46, and the side surface 64 extends transverse to the outer face 50 and extends from the outer face 50 to the bottom surface 62. Although it is shown that the recess 60 has a circular profile, it is contemplated that the recess 60 could have any shaped profile, including triangular, rectangular, or any other multiple-sided configuration.

The trunk portion 48 includes an inner wall 56, an outer wall 54, an end wall 58, and an opening 66. The outer wall 54 is sized to fit within a pertuator hole left within the skull 10 following surgery. The inner wall 56 is co-axial with the outer wall 54 and extends transverse to the bottom surface 62 of the recess 60 from the bottom surface 62 to the end wall 58 of the trunk portion 48. The end wall 58 runs parallel to the outer face 50. An opening 66 extends co-axially with the recess 60 and is defined between the bottom surface 62 of the recess 60 and the end wall 58 of the trunk portion 48. The opening 66 is circular in nature and generally of a smaller diameter than the recess 60.

As shown in FIG. 3B, the flange portion 46 further includes a plurality of expansion slots 70 extending from the periphery of the recess 60 to the periphery of the flange portion 46. The expansion slots 70 are 0.01″ to 0.05″ wide, extend transverse to the outer face 50 of the flange portion 46, and are defined from the outer face 50 to the inner face 52. Although six expansion slots 70 are depicted in the drawing, it is contemplated that any number of slots 70 could be utilized. As will be further explained later, the expansion slots 70 allow the flange portion 46 to expand radially outward from the distal end 67 of the plate 22 to provide axial compression at the bone fixation site to aid in securing the bones together. The expansion slots 70 accomplish this by allowing the flange portion 46 to expand as a series of flange sections 71 instead of as a single flange. This configuration allows the flange portion 46 to provide a larger, and more stable, surface area of axial compression to the skull 10 and the skull fragment 14, as well as allowing the flange portion 46 to more precisely follow the contour of the skull 10 and the skull fragment 14.

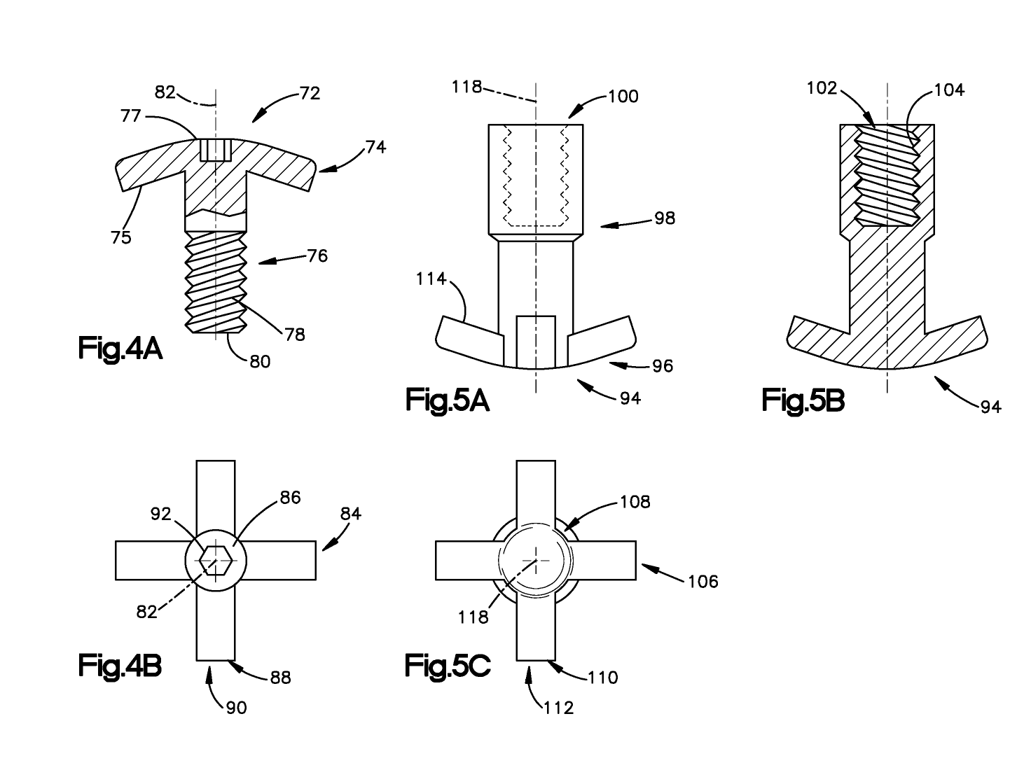

FIG. 4A depicts a screw 72 used in securing the bones together. The screw 72 comprises a head portion 74 and a shank portion 76. The shank portion 76 is sized to fit through the recess 38 and opening 44 of the first plate 20. The head portion 74 is sized to fit into the recess 38 of the first plate 20, but not through the opening 44. The shank portion 76 includes an external thread convolution 78 extending from a leading end 80 of the shank 76 towards the head portion 74. The external thread convolution 78 may be any thread configuration known in the art; for example, helical, square, or left-handed. The external thread convolution 78 is rotatable about a longitudinal central axis 82 of the screw 72.

As shown in FIG. 4B, screw 72 includes a plurality of resiliently bendable sections 84 extending generally radially outward from a central portion 86 of the head portion 74. The bendable sections 84 can be made of any material that allows them to bend under an axial force without plastically deforming. However, it is preferable that the bendable sections 84 (and thus the entire screw 72) be made of titanium because the screw 72 must be strong enough to sustain the loads required to affix the bones together. Each of the bendable sections 84 may be rectangular in nature or may taper inwards or outwards from the central portion 86 to form triangular or trapezoidal configurations. In addition, the bendable sections 84 extend from the central portion 86 of the head portion 74 in a direction toward the leading end 80 (FIG. 4A) of the shank portion 76 when the screw 72 is in an initial condition (FIG. 4A). Each of the bendable sections 84 includes an end portion 88 at a distal end 90 and a bottom surface 75. The end portions 88 extend transverse to the bendable sections 84 and may be orthogonal to the bendable sections 84 or extend at an angle. Furthermore, the end portion 88s may each be a series of teeth in a saw, or similar, pattern. In such a case, the side surface 42 of the first plate 20 will include mating teeth.

Head portion 74 of screw 72 further includes structure 92, located in the central portion 86, for receiving a tool for rotating the screw 72 about the longitudinal central axis 82. The structure 92 may be, but is not limited to, receiving means for any standard screwdriver or allen wrench, including phillips head, flat head, hex head, or the like.

As shown in FIG. 5A, a mating nut 94 is disclosed having a head portion 96 and a body portion 98. The body portion 98 is sized to fit through the recess 60 and opening 66 of the second plate 22. The head portion 96 is sized to fit into the recess 60 of the second plate 22, but not through the opening 66. The body portion 98 includes an opening 102 (FIG. 5B) that extends from a leading end 100 towards the head portion 96. The opening 102 includes an internal thread convolution 104 extending from the leading end 100 of the body 98 towards the head portion 96 along a central axis 118. The internal thread convolution 104 may be any thread configuration known in the art; for example, helical, square, or left-handed. However, the internal thread convolution 104 of the nut 94 must mate with the external thread convolution 78 of the screw 72.

As shown in FIG. 5C, nut 94 includes a plurality of resiliently bendable sections 106 extending generally radially outward from a central portion 108 of the head portion 96. The bendable sections 106 can be made of any material that allows them to bend under an axial force without plastically deforming. However, it is preferable that the bendable sections 106 (and thus the entire nut 94) be made of titanium because the nut 94 must be strong enough to sustain the loads later discussed in order to affix the bones together. Each of the bendable sections 106 may be rectangular in nature or may taper inwards or outwards from the central portion 108 to form triangular or trapezoidal configurations. In addition, the bendable sections 106 extend from the central portion 108 of the head portion 96 in a direction toward the leading end 100 (FIG. 5A) of the body portion 98 when the nut 94 is in an initial condition (FIG. 5A). Each of the bendable sections 106 includes an end portion 110 at a distal end 112 and a bottom surface 114. The end portions 110 extend transverse to the bendable sections 106 and may be orthogonal to the bendable sections 106 or extend at an angle. Furthermore, the end portions 110 may each be a series of teeth in a saw, or similar, pattern. In such a case, the side surface 64 of second plate 22 will include mating teeth.

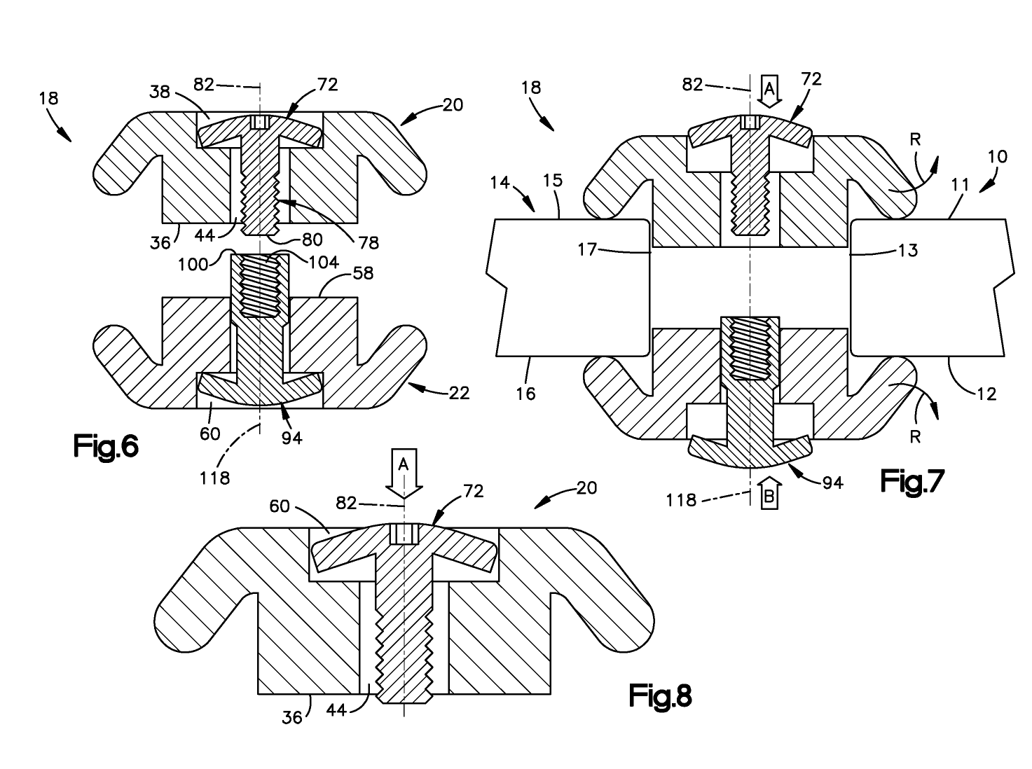

According to one embodiment of the present invention, the first plate 20 and second plate 22 are oriented with their end walls 36, 58 facing each other. The shank portion 76 of the screw 72 is inserted through the recess 38 and opening 44 of the first plate 20. Likewise, the body portion 98 of the nut 94 is inserted through the recess 60 and opening 66 of the second plate 22 as shown in FIG. 6 so that the external thread convolution 78 of the screw 72 and the opening 102 of the nut 94 are in proximity with each other. This makes the inner face 30 of the first plate 20 substantially parallel to the inner face 52 of the second plate 22, and the recess 38 and opening 44 of the first plate 20 become coaxial with the recess 60 and opening 66 of the second plate 22. As the shank portion 76 of the screw 72 is moved closer to the body portion 98 of the nut 94, the head portion 74 of the screw 72 moves into the recess 38 of the first plate 20. The initial condition of the resiliently bendable sections 84 on the head portion 74 of the screw 72 allows the entire head portion 74 of the screw 72 to fit within the recess 38 of the first plate 20. Likewise, as the body portion 98 of the nut 94 is moved closer to the shank portion 76 of the screw 72, the head portion 96 of the nut 94 moves into the recess 60 of the second plate 22. The initial condition of the resiliently bendable sections 106 on the head portion 96 of the nut 94 allows the entire head portion 96 of the nut 94 to fit within the recess 60 of the second plate 22.

As shown in FIG. 7, the inner face 30 of the first plate 20 and the inner face 52 of the second plate 22 cooperate to secure the removed skull fragment 14 to the intact skull 10. The apparatus 18 is positioned such that a portion of the intact skull 10 lies in between the inner wall 34 of the first plate 20 and the inner wall 56 of the second plate 22. In this positioning, the outer wall 32 of the trunk portion 26 of the first plate 20 should be aligned such that it contacts the side wall 13 of the skull 10. The outer wall 54 of the trunk portion 48 of the second plate 22 should also be aligned such that it contacts that side wall 13 of the skull 10. Furthermore, a portion of the skull fragment 14 to be secured back to the skull 10 is positioned to lie between the flange portion 24 of the first plate 20 and the flange portion 46 of the second plate 22. In this positioning, the outer wall 32 of the trunk portion 26 of the first plate 20 should be aligned such that it contacts the side wall 17 of the skull fragment 14. The outer wall 54 of the trunk portion 48 of the second plate 22 should also be aligned such that it contacts that side wall 17 of the skull fragment 14. The skull fragment 14 and the intact skull 10 lie 180° from each other relative to the opening 44 in the first plate 20 and the opening 66 of the second plate 22.

The first plate 20 and screw 72 are then moved in the direction ‘A’ indicated until the external thread configuration 78 on the screw 72 moves into the opening 102 of the nut 94. The screw 72 is then rotated along its longitudinal central axis 82 using a tool (not shown) to engage the external thread configuration 78 of the screw 72 with the internal thread configuration 104 of the nut 94. As the screw 72 is further rotated, the increased engagement between the threads 78, 104 decreases the axial distance between the inner face 30 of the first plate 20 and the inner face 56 of the second plate 22, causing the first plate 20 to move in the direction ‘A’ indicated, and the second plate 22 to move in the direction ‘B’ indicated. This decreased distance initially brings the flange portion 24 of the first plate 20 into contact with the top 11 of the skull 10 and the top 15 of the skull fragment 14 and the flange portion 46 of the second plate 22 into contact with the bottom 12 of the skull 10 and the bottom 16 of the skull fragment 14. Then, as the plates 20, 22 continue to move closer together, the flange portion 24 of the first plate 20 and the flange portion 46 of the second plate 22 begin to expand radially outward in the direction ‘R’ as a moment is created between the flange portion and the trunk portion of each plate since the geometry of the trunk portion allows it to move through the hole between the skull 10 and the skull fragment 14, while the flange portion is incapable of such movement. This radial expansion of the flange portion 24 of the first plate 20 and the flange portion 46 of the second plate 22 is facilitated by the expansion slots 68, 70 on each (FIGS. 2B & 3B). The expansion slots 68, 70 allow each flange to react to the moment by radially expanding it as a series of flange sections rather that as a single unit. Attempting to radially expand each flange portion as a single unit would create large stress concentrations around the periphery of each recess 38, 60 in reaction to the collective moment created by the skull fragment 14 and skull 10 acting on the periphery of the flange portion as the trunk portion moves through the hole between the skull 10 and skull fragment 14. Adding the expansion slots allows each flange section to shoulder a fraction of the moment on the entire flange portion, which reduces the stress concentration around the periphery of the recess by eliminating stresses that would otherwise arise between sections, and allows each section to adapt to its respective moment and more precisely conform to the contour of the particular skull fragment 14 or skull 10 it is in contact with.

Furthermore, the outer wall 32 of the trunk portion 26 of the first plate 20 moves along the side wall 13 of the skull 10 and the side wall 17 of the skull fragment 14. Likewise, the outer wall 54 of the trunk portion 48 of the second plate 22 moves along the side wall 13 of the skull 10 and the side wall 17 of the skull fragment 14.

As shown in FIG. 8, continued rotation of the screw 72 first causes the end portions 88 of the bendable sections 84 of the screw 72 to contact the bottom surface 40 of the recess 38 of the first plate 20. Then, the end portions 88 of the bendable sections 84 slide radially outward from the longitudinal central axis 82 along the bottom surface 40 of the recess 38 until the end portions 88 contact the side surface 42 of the recess 38. As the end portions 88 slide along the bottom surface 40, the bendable sections 84 bend relative to the head portion 74 of the screw 72 due to the shank portion 76 moving in the direction ‘A’ indicated.

Continued rotation of the screw 72 simultaneously causes the end portions 110 of the bendable sections 106 of the nut 94 to contact the bottom surface 62 of the recess 60 of the second plate 22. Then, the end portions 110 of the bendable sections 106 slide radially outward from the central axis 118 along the bottom surface 62 of the recess 60 until the end portions 110 contact the side surface 64 of the recess 60. As the end portions 110 slide along the bottom surface 62, the bendable sections 106 bend relative to the head portion 96 of the nut 94 due to the body portion 98 moving in the direction ‘B’ as indicated in FIG. 7.

As the screw 72 moves in the direction ‘A’ indicated, the top 77 of the head portion 74 of the screw 72 moves closer to the bottom surface 40 of the recess 38 until the top 77 of the head portion 74 is closer to the bottom surface 40 of the recess 38 than the outer face 28 of the flange portion 24 is. This movement minimizes the resulting protrusion the patient will have once the skin is later pulled back over the exposed scalp to finish the surgery.

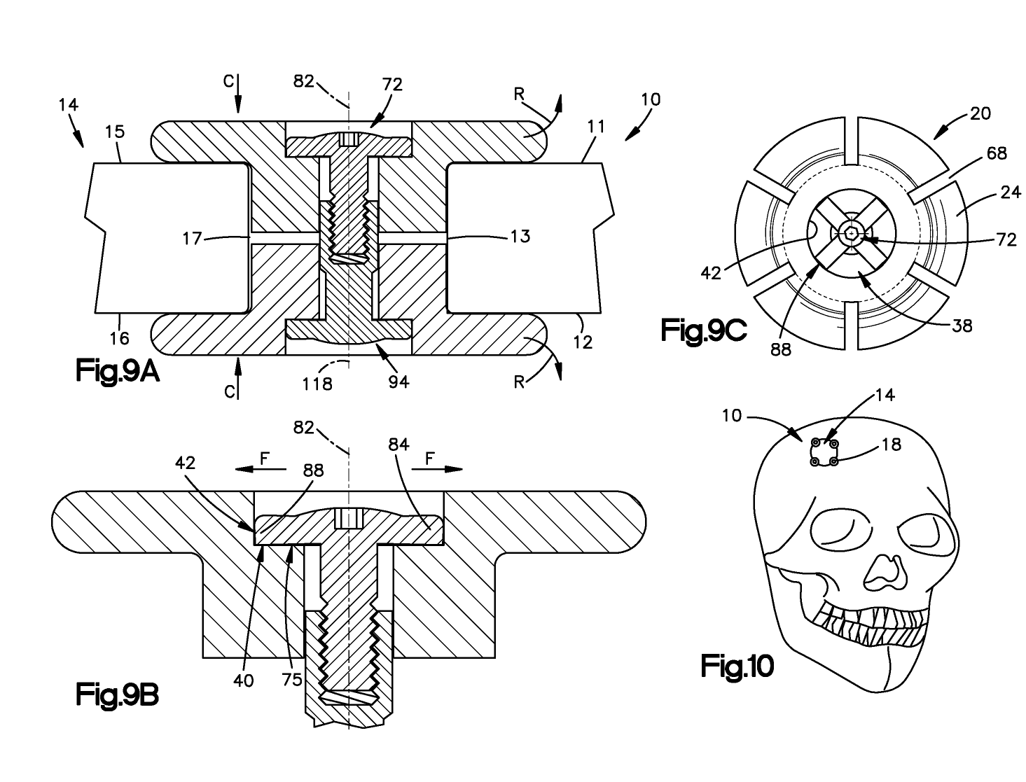

FIG. 9A depicts the apparatus 18 in its final, locked position. In this configuration, the flange portion 24 of the first plate 20 has expanded radially outward in the direction ‘R’ sufficiently enough such that the inner face 30 of the flange portion 24 of the first plate 20 is parallel to, and in contact with, the top surface 11 of the skull 10 and the top surface 15 of the skull fragment 14. Likewise, the flange portion 46 of the second plate 22 has expand radially outward in the direction ‘R’ sufficiently enough such that the inner face 52 of the flange portion 46 of the second plate 22 is parallel to, and in contact with, the bottom surface 12 of the skull 10 and the bottom surface 16 of the skull fragment 14. The screw 72 has been tightened to the nut 94 sufficiently enough to cause the first plate 20 and second plate 22 to apply an axial clamping force ‘C’ to the skull 10 and skull fragment 14. The axial clamping force ‘C’ is derived from the threaded connection between the screw 72 and the nut 94. This connection causes the bottom surface 75 of the head portion 74 of the screw 72 to apply an axial force downward against the bottom surface 40 of the recess 38 of the first plate 20, which in turn causes the inner face 30 of the flange portion 24 of the first plate 20 to apply a downward force to the top surfaces 11, 15 of the skull 10 and the skull fragment 14. Likewise, this connection simultaneously causes the bottom surface 114 of the head portion 96 of the nut 94 to apply an axial force upward against the bottom surface 62 of the recess 60 of the second plate 22, which in turn causes the inner face 52 of the flange portion 46 of the second plate 22 to apply an upward force to the bottom surfaces 12, 16 of the skull 10 and the skull fragment 14. Furthermore, the geometry of the first plate 20 and second plate 22 will provide additional axial clamping force in the direction ‘C’. Since the flange portion 24 of the first plate 20 and the flange portion 46 of the second plate 22 have extended radially outward during installation as described above, the flange portions 24, 46 have been elastically deformed from their initial conditions. The tendency of the flange portion 24 of the first plate 20 and the flange portion 46 of the second plate 22 to return to its initial condition, i.e. extending in a direction toward the distal end 45 of the first plate 20 and the distal end 67 of the second plate 22, will cause the inner face 30 of the first plate 20 and inner face 52 of the second plate 22 to apply a clamping force in the direction ‘C’. The combination of these axial clamping forces rigidly fixes the skull fragment 14 to the skull 10 and prevents relative movement between the two.

As shown in FIG. 9B, the nature of the bendable sections 84 of the screw 72 also provide a radial force ‘F’ to prohibit the screw 72 from unscrewing following implantation. The bending of the bendable sections 84 during implantation to the point that the end portions 88 are in contact with the side surface 42 of the recess 38 creates a radial force outward against the side surface 42. As the shank portion 76 of the screw 72 is further pulled through the opening 44 due to increased thread engagement between the screw 72 and the nut 94, the end portions 88 of the bendable sections 84 are forced radially outward due to the bending moment created, but since the side surface 42 of the recess 38 is present, the end portions 88 of the bendable sections 84 cannot extend farther and therefore apply an increasing outward radial force to the side surface 42 as the screw 72 is further engaged with the nut 94 until complete installation has occurred. At that point, the end portions 88 of the bendable sections 84 apply a continuous outward radial force ‘F’ to the side surface 42 of the recess 38.

In a similar fashion (not shown), the end portions 110 of the bendable sections 106 apply the same outward radial force to the side surface 64 of the recess 60 of the second plate 22 during implantation. As the body portion 98 of the nut 94 is further pulled through the opening 66 due to increased thread engagement between the screw 72 and the nut 94, the end portions 110 of the bendable sections 106 are forced radially outward due to the bending moment created, but since the side surface 64 of the recess 60 is there, the end portions 110 of the bendable sections 106 cannot extend farther and therefore apply an increasing radial force to the side surface 64 as the screw 72 is further engaged with the nut 94 until complete installation has occurred. At that point, the end portions 110 of the bendable sections 106 apply a continuous outward radial force to the side surface 64 of the recess 60 of the second plate 22. The outward radial force ‘F’ created by the bendable sections 84 of the screw 72 coupled with the outward radial force ‘F’ created by the bendable sections 110 of the nut 94 aid in prohibiting rotation of the screw 72 relative to the nut 94 after the device 18 has been installed.

Since a common problem with bone fixation devices is the unscrewing of the bone fixation screw over time due to vibration or physical activity of the patient, the outward radial force ‘F’ present in the current invention prohibits the unscrewing of the screw 72 from the nut 94. The outward radial force ‘F’ creates friction between the end portion 88 of the bendable sections 84 and the side surface 42 of the recess 38 of the first plate 20, and between the end portion 110 of the bendable sections 106 and the side surface 64 of the recess 60 in the second plate 22. That friction force becomes high enough upon implantation that vibration or physical activity of the patient will not create enough force to overcome the frictional force and loosen the screw 72 relative to the nut 94, thus creating a longer lasting and safer bone fixation device 18 that can more adequately effectuate bone healing and regeneration.

FIG. 9C depicts a top view of the end portions 88 of the bendable sections 84 of the screw 72 applying the outward radial force ‘F’ to the side surface 42 of the recess 38 of the first plate 20. As noted supra, each end portion 88 could include a series of teeth in a saw pattern, with the side surface 42 of the recess 38 of the first plate 20 including a mating series of teeth. If that is the case, the teeth on the end portions 88 provide a greater surface area that contacts the side surface 42 of the recess 38. That increased surface area increases the frictional force generated between the bendable sections 84 and the side surface 42, thus further resisting the tendency of the screw 72 to unscrew from the nut 94. It is understood that a similar mating tooth feature could be incorporated into the bendable sections 110 of the nut 94 and the side surface 64 of the second plate 22 to further resist the tendency of the nut 94 to unscrew from the screw 72.

FIG. 10 depicts a skull fragment 14 secured to the skull 10 utilizing four of the devices 18 of the present invention. Although the drawing illustrates the use of four bone attachment devices 18, it will be understood to those in the art that any number of bone attachment devices 18 could be used in securing the bone fragment 14 back to the skull 10.

A further embodiment in accordance with the present invention is depicted in FIGS. 11-17. A jaw bone disposed in a patient's body has been illustrated schematically in FIG. 11. As shown in the exploded view of FIG. 12, the present invention 200 utilizes a base portion 210 and a fastener portion 240 to provide a stable site within the jaw to receive a dental implant, such as a replacement tooth.

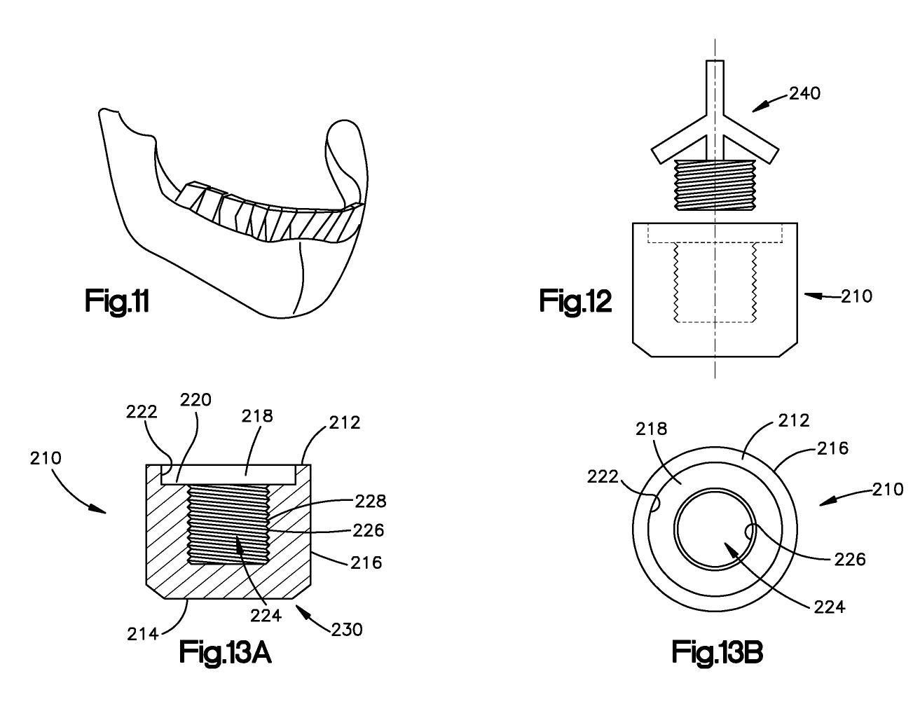

FIGS. 13A-B depict the base portion 210 for creating a secured dental implant site. The base 210 is substantially circular in nature. The base 210 is defined by a top surface 212, an end wall 214, and an outer wall 216 extending therebetween. The top surface 212 includes a recess 218 defined from the top surface towards the end wall 214. The recess 218 has a bottom surface 220 and a side surface 222. The bottom surface 220 is parallel to the top surface 212 of the base 210, and the side surface 222 extends transverse to the top surface and extends from the top surface to the bottom surface 220. Although it is shown that the recess 218 has a circular profile, it is contemplated that the recess could have any shaped profile, including triangular, rectangular, or any other multiple-sided configuration.

An opening 224 extends co-axially with the recess 218 and is defined between the bottom surface 220 of the recess 218 and a point between the bottom surface of the recess and the end wall 214. The opening 224 defines an inner wall 226 that is circular in nature and generally of a smaller diameter than the recess 218. The inner wall 226 includes an internal thread convolution 228 extending the depth of the opening 224. The inner wall 226 is generally concentric with the outer wall 216

The base 210 is secured, or otherwise affixed, at a desired location along the jaw line of the patient where the dental implant is required (not shown). The base 210 is secured to the jaw such that the top surface 212 of the base 210 is substantially aligned with the top of the opening made in the jaw bone to receive the base 210. This orientation will make the opening 224 and recess 218 of the base 210 visible from inside of the mouth of the patient.

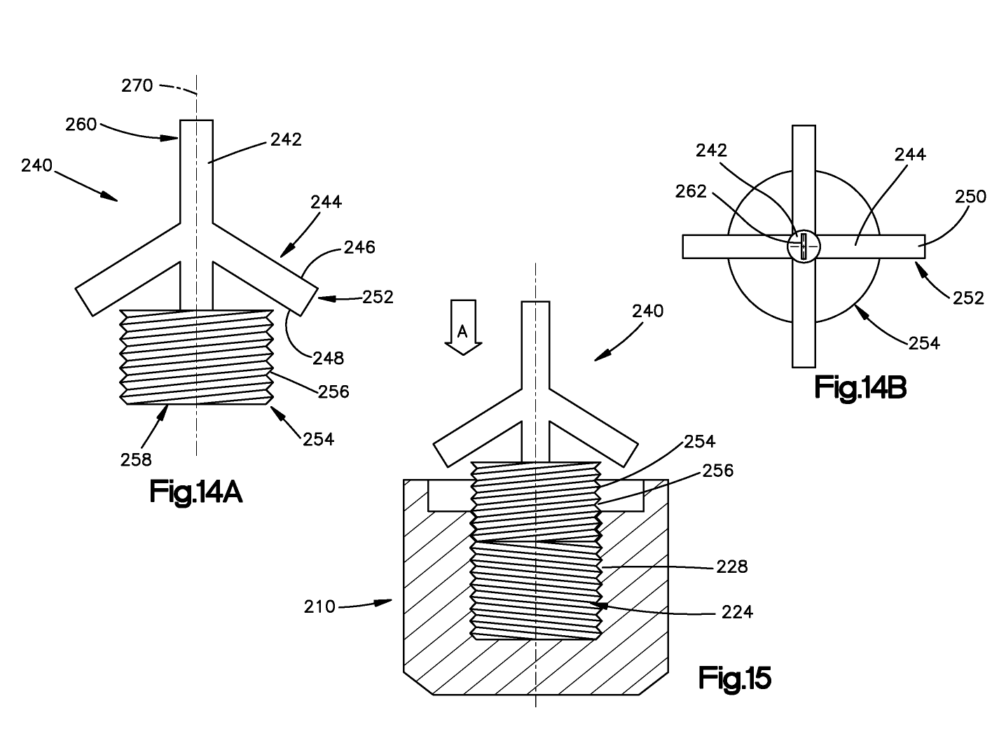

FIGS. 14A-B depict the fastener portion 240 used in securing the dental implant site. The fastener portion 240 comprises a pin 242 and a trunk 254. The trunk 254 is sized to fit through the recess 218 of the base 210 and into the opening of the base. The pin 242 is substantially smaller in cross-section than the trunk 254. Both the trunk 254 and the pin 242 are generally circular in profile. The pin 242 is sized to accommodate a dental implant such as a replacement tooth once the device 200 is implanted within the patient's jaw. The trunk 254 includes an external thread convolution 256 extending from a distal end 258 of the trunk 254 towards the pin 242. The external thread convolution 256 may be any thread configuration known in the art; for example, helical, square, or left-handed. The external thread convolution 256 is rotatable about a longitudinal central axis 270 of the fastener portion 240.

Pin 242 includes a plurality of resiliently bendable sections 244 extending generally radially outward from the pin 242. The bendable sections 244 can be made of any material that allows them to bend under an axial force without plastically deforming. However, it is preferable that the bendable sections 244 (and thus the fastener portion 240) be made of titanium because the fastener portion 240 must be strong enough to sustain the loads required to maintain a dental implant within the jaw once implanted for everyday use. Each of the bendable sections 244 may be rectangular in nature or may taper inwards or outwards from the pin 242 to form triangular or trapezoidal configurations. In addition, the bendable sections 244 extend from the pin 242 in a direction toward the distal end 258 of the trunk 254 when the fastener portion 240 is in an initial condition (FIG. 14A). Each of the bendable sections 244 includes an end portion 250 at a distal end 252, a top surface 246, and a bottom surface 248. The end portions 250 extend transverse to the bendable sections 244 and may be orthogonal to the bendable sections 244 or extend at an angle. Furthermore, the end portions 250 may each be a series of teeth in a saw, or similar, pattern. In such a case, the side surface 222 of the base portion 210 will include mating teeth.

A head portion 260 of the post 242 includes structure 262 for receiving a tool for rotating the fastener 240 about a longitudinal central axis 270. The structure 262 may be, but is not limited to, receiving means for any standard screwdriver or allen wrench, including phillips head, flat head, hex head, or the like.

According to one embodiment of the present invention, once the base 210 is secured within the jaw, the trunk 254 of the fastener portion 240 is inserted through the recess 218 and into the opening 224 of the base portion 210 such that the external thread convolution 256 of the trunk 254 and the internal thread convolution 228 of the opening 224 of the base 210 are in proximity with each other. The fastener portion 240 is then rotated along the longitudinal central axis 270 using a tool (not shown) to engage the external thread configuration 256 of the trunk 254 with the internal thread configuration 228 of the base 210. As the fastener 240 is further rotated, the increased engagement between the threads 228, 256 decreases the axial distance between the bendable sections 244 and the recess 218 in the base 210, causing the fastener portion 240 to move in the direction ‘A’ indicated.

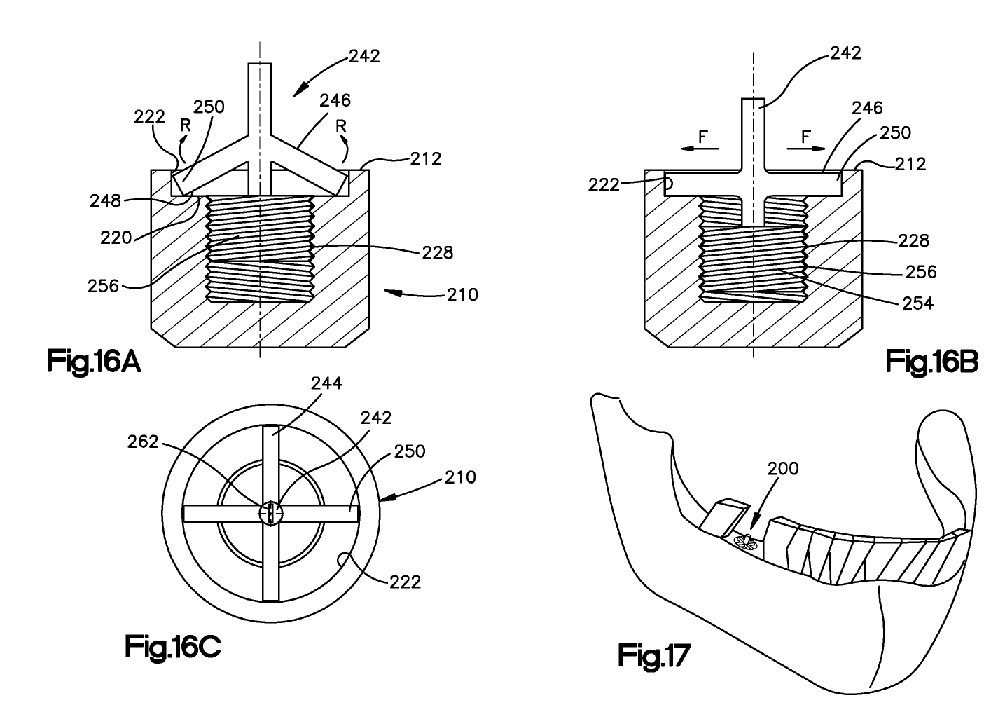

Continued rotation of the fastener 240 first causes the end portions 250 of the bendable sections 244 of the post 242 to contact the bottom surface 220 of the recess 218 of the base 210. As the post 242 moves closer to the base 210 (FIG. 16A), the bendable sections 244 begin to expand radially outward in the direction ‘R’ as a moment is created between the post 242 and the bendable sections 244 since the geometry of the fastener 240 allows the trunk 254 to pass through the recess 218, while the bendable sections 244 are incapable of such movement. The end portions 250 of the bendable sections 244 likewise slide radially outward from the longitudinal central axis 270 along the bottom surface 220 of the recess 218 until the end portions 250 contact the side surface 222 of the recess 218.

As the fastener portion 240 moves in the direction ‘A’ indicated, and the bendable sections 244 slide radially outward, the top surface 246 of each bendable section 244 moves closer to the bottom surface 220 of the recess 218 until the top surface 246 of each bendable section 244 is closer to the bottom surface 220 of the recess 218 than the top surface 212 of the base 210 is.

FIG. 16A-B depicts the apparatus in its final, locked position. In this configuration, the bendable sections 244 have expanded radially outward in the direction ‘R’ sufficiently enough such that the bottom surface 248 of each bendable section 244 is parallel to, and in contact with, the bottom surface 220 of the recess 218 of the base 210.

The nature of the bendable sections 244 provides a radial force ‘F’ to prohibit the fastener portion 240 from unscrewing following implantation. The bending of the bendable sections 244 during implantation to the point that the end portions 250 are in contact with the side surface 222 of the recess 218 creates a radial force outward against the side surface 222. As the trunk 254 increasingly threadably engages the base 210, the end portions 250 of the bendable sections 244 are forced radially outward due to the bending moment created, but since the side surface 222 of the recess 218 is present, the end portions 250 of the bendable sections 244 cannot extend farther and therefore apply an increasing outward radial force to the side surface 222 as the fastener portion 240 is further engaged with the base 210 until complete installation has occurred. At that point, the end portions 250 of the bendable sections 244 apply a continuous outward radial force ‘F’ to the side surface 222 of the recess 218.

This fastening configuration ensures that once the desired dental implant has been affixed to the post 242 on the fastener portion 240 of the device 200, the dental implant will not rotate relative to the base 210 or jaw, and thus will not become loose due to vibration or otherwise daily use of the jaw by the patient in chewing, talking, etc. This will help prevent discomfort and possible injury from loosened replacement teeth following implantation.

From the above description of the invention, those skilled in the art will perceive improvements, changes and modifications. Such improvements, changes and modifications within the skill of the art are intended to be covered by the appended claims.

Having described the invention, the following is claimed:

1. An apparatus for use in attaching bone in a patient's body, said apparatus comprising:
first and second plates, each of said plates having a flange portion and a trunk portion, said flange portion having an outer face and an inner face, said trunk portion having an outer wall and an end, said flange portion at least partially defining a recess, said first and second plates having an opening which extends from said recess to said end wall of said trunk portion;
a nut having a head portion and a body portion, said body portion having an opening, said opening having an internal thread convolution, said head portion having a plurality of resiliently bendable sections which extend outward from said head portion;
a screw having a head portion and a shank portion, said shank portion having an external thread convolution which is engageable with said internal thread convolution of said nut, said head portion having a plurality of resiliently bendable sections which extend outward from said head portion, said screw being rotatable to turn said external thread convolution on said shank portion into engagement with said internal thread convolution of said nut; and
said bendable sections of said screw being movable into engagement with said recess of said first plate upon rotation of said screw, wherein further rotation of said screw retains said bendable sections in said recess of said first plate against rotation relative to said first plate, said bendable sections of said nut moving into engagement with said recess of said second plate as said nut becomes engaged with said screw, said bendable sections of said nut becoming retained in said recess of said second plate against rotation relative to said second plate, said bendable sections of said screw and said bendable sections of said nut providing radial compression to said first and second plate upon engagement to secure bones together.

2. The apparatus of claim 1 wherein each of said recesses includes a bottom surface which is spaced from said inner face and a side surface which extends transverse to said outer face and to said bottom surface of said recess.

3. The apparatus of claim 1 wherein said bendable sections of said screw extend outward from a central portion of said head portion of said screw.

4. The apparatus of claim 3 wherein each of said bendable sections has an end portion disposed at a distal end.

5. The apparatus of claim 4 wherein said bendable sections further extend in a direction toward said leading end of said shank portion when said screw is in an initial condition.

6. The apparatus of claim 3 wherein said central portion includes structure means for receiving a tool for rotating said screw.

7. The apparatus of claim 1 wherein said bendable sections of said nut extend outward from a central portion of said head portion of said nut.

8. The apparatus of claim 7 wherein each of said bendable sections has an end portion disposed at a distal end.

9. The apparatus of claim 8 wherein said bendable sections further extend in a direction toward said leading end of said shank portion when said screw is in an initial condition.

10. The apparatus of claim 1 wherein said bendable sections of said screw move into sliding engagement with a bottom surface of said recess of said first plate.

11. The apparatus of claim 10 wherein end portions of said bendable sections subsequently move into engagement with a side surface of said recess of said first plate as said bendable sections are deflected to retain said screw against rotation relative to said first plate.

12. The apparatus of claim 1 wherein said bendable sections of said nut move into sliding engagement with a bottom surface of said recess of said second plate.

13. The apparatus of claim 12 wherein end portions of said bendable sections subsequently move into engagement with a side surface of said recess of said second plate as said bendable sections are deflected to retain said nut against rotation relative to said second plate.

14. The apparatus of claim 1 wherein said screw is rotatable about a longitudinal central axis of said shank portion.

15. The apparatus of claim 1 where the flange portion includes a plurality of expansion slots.

16. The apparatus of claim 15, wherein the expansion slots extend from the periphery of the recess to the periphery of the flange portion, and are defined from the outer face to the inner face of the flange portion.

17. An apparatus for use in attaching bone in a patient's body, said apparatus comprising:
first and second plates, each of said plates having a flange portion and a trunk portion, said flange portion having an outer face and an inner face, said trunk portion having an outer wall and an end, said flange portion at least partially defining a recess extending from said outer face toward said inner face, said recess having a bottom surface which is spaced from said inner face, said recess further having a side surface which extends transverse to said outer face and to said bottom surface of said recess, said first and second plates having an opening which extends from said bottom surface of said recess to said end wall of said trunk portion;
a nut having a head portion and a body portion, said body portion having a opening at a leading end, said opening having an internal thread convolution extending from said leading end of said body portion towards said head portion, said head portion having a plurality of resiliently bendable sections which extend outward from a central portion of said head portion in a direction toward said leading end of said body portion when said nut is in an initial condition, each of said resiliently bendable sections having an end portion disposed at a distal end; and
a screw having a head portion and a shank portion, said shank portion having an external thread convolution extending from a leading end of said shank portion towards said head portion which is engageable with said internal thread convolution of said nut, said head portion having a plurality of resiliently bendable sections which extend outward from a central portion of said head portion in a direction toward said leading end of said shank portion when said screw is in an initial condition, each of said resiliently bendable sections having an end portion disposed at a distal end, said screw being rotatable about a longitudinal central axis of said shank portion to turn said external thread convolution on said shank portion into engagement with said internal thread convolution of said nut, said resiliently bendable sections of said screw moving into engagement with said bottom surface of said recess of said first plate and are deflected in a direction away from the leading end of said shank portion of said screw as said screw is rotated about said longitudinal central axis, said end portions of said resiliently bendable sections of said screw move into engagement with said side surface of said recess of said first plate as said sections are deflected to retain said screw against rotation relative to said first plate, said resiliently bendable sections on said nut moving into sliding engagement with said bottom surface of said recess of said second plate and are deflected in a direction away from the leading end of said body potion of said nut as said nut becomes engaged with said screw, said end portions of said resiliently bendable sections of said nut move into engagement with said side surface of said recess of said second plate as said sections are deflected to retain said nut against rotation relative to said second plate, said end portions of said resiliently bendable sections of said screw and said end portions of said resiliently bendable sections of said nut providing radial compression to said side surface of said first and second plate upon engagement of said screw and said nut to secure bones together.

18. The apparatus of claim 17 where the flange portion includes a plurality of expansion slots.

19. The apparatus of claim 18, wherein the expansion slots extend from the periphery of the recess to the periphery of the flange portion, and are defined from the outer face to the inner face of the flange portion.

20. A method of attaching bone in a patient's body comprising the steps of:
placing a first plate near a top surface of a bone and a bone fragment, the first plate at least partially defining a recess, the first plate having an opening which extends from the recess to an end wall of the first plate;
placing a second plate near a bottom surface of the bone and the bone fragment, the second plate at least partially defining a recess, the second plate having an opening which extends from the recess to an end wall of the second plate;
inserting a distal end of a screw through the opening of the first plate, the screw having an external thread convolution at the distal end and bendable sections at a proximal end;
inserting a distal end of a nut through the opening of the second plate, the nut having an internal thread convolution at the distal end and bendable sections at a proximal end;
rotating the screw to engage the external thread convolution of the screw with the internal thread convolution of the nut, wherein further rotation of the screw clamps the bone and the bone fragment between the first plate and the second plate;
retaining the bendable sections of the screw against the recess of the first plate, which prevents further rotation of the screw relative to the first plate; and
retaining the bendable sections of the nut against the recess of the second plate, which prevents further rotation of the nut relative to the second plate.

21. The method of claim 20 wherein the step of retaining the bendable sections of the screw comprises moving end portions of the bendable sections of the screw into engagement with a side surface of the recess of the first plate.

22. The method of claim 20 wherein the step of retaining the bendable sections of the nut comprises moving end portions of the bendable sections of the nut into engagement with a side surface of the recess of the second plate.

23. An apparatus for providing a dental implant site, said apparatus comprising:
a base having a top surface, an end wall, and a side wall, said top surface at least partially defining a recess, said base further comprising an opening which extends from said recess toward said end wall, said opening defining an inner wall having an internal thread convolution,
a fastener having a post and a trunk, said trunk having an external thread convolution which is engageable with said internal thread convolution of said base, said post having a plurality of resiliently bendable sections which extend outward from said post, said fastener being rotatable to turn said external thread convolution on said trunk into engagement with said internal thread convolution of said base; and
said bendable sections on said post being movable into engagement with said recess of said base upon rotation of said fastener, wherein further rotation of said fastener retains said bendable sections in said recess of said base against rotation relative to said base, said bendable sections of said fastener providing radial compression to said base upon engagement to provide an attachment site for a dental implant.

24. The apparatus of claim 23 wherein said recess includes a bottom surface which is spaced from said top surface and a side surface which extends transverse to said side wall of said base and to said bottom surface of said recess.

25. The apparatus of claim 23 wherein each of said bendable sections has an end portion disposed at a distal end.

26. The apparatus of claim 25 wherein said bendable sections extend in a direction toward a distal end of said trunk when the fastener portion is in an initial condition.

27. The apparatus of claim 23 wherein said post includes structure means for receiving a tool for rotating said fastener portion.

28. The apparatus of claim 23 wherein said bendable sections of said fastener move into sliding engagement with a bottom surface of said recess of said base.

29. The apparatus of claim 28 wherein end portions of said bendable sections subsequently move into engagement with a side surface of said recess of said base as said bendable sections are deflected to retain said fastener against rotation relative to said base.

30. The apparatus of claim 23 where said fastener is rotatable about a longitudinal central axis of said post.
